(19) United States
(12) Patent Application Publication
(10) Pub. No.: US 2012/0291186 A1
(43) Pub. Date: Nov. 22, 2012

(54) Collapsible goggles

(76) Inventor: Chun-Chih CHENG, Tainan City (TW)

(21) Appl. No.: 13/068,521

(22) Filed: May 16, 2011

(51) Int. Cl. A61F 9/02

(52) U.S. Cl. 2/431

(57) ABSTRACT

A pair of collapsible goggles consists of a flexible frame and an eyeglass. The flexible frame has plural engaging grooves and plural air holes formed around the edge. The eyeglass is formed curved, provided with engaging projections. A connecting arm is installed at two sides of the eyeglass respectively. In assembly, one end of the flexible frame is first fitted in the curved recessed surface of the eyeglass, with the engaging projections of the eyeglass inserted in the engaging grooves one by one to enable the flexible frame extended straight against the eyeglass. Next, the connecting arms are put at two sides of the eyeglass to engage with the positioning holes. So the collapsible goggles are easily and stably assembled together.

### BACKGROUND OF THE INVENTION

1. Field of the Invention

This invention relates to a pair of collapsible goggles, particularly to one provided with a flexible frame stably assembled together with an eyeglass with a smooth appearance, able to effectively protect eyes from being hurt by foreign objects.

2. Description of the Prior Art

Commonly, a pair of conventional goggles is composed of a rigid eyeglass and a semi-rigid frame, with the eyeglass forcefully wrapped around the semi-rigid frame. In order to enable the eyeglass to be fitted in the frame, the frame is usually manufactured with a slightly larger size, apt to result in a poor combination between the eyeglass and the frame. Moreover, molding and finishing costs of the conventional goggles stay at a high level.

### SUMMARY OF THE INVENTION

The object of this invention is to offer a pair of collapsible goggles provided with a frame and an eyeglass assembled together stably with an integral smooth appearance.

The main characteristics of the invention are a flexible frame and an eyeglass. The flexible frame is formed mostly waved, provided with a fitting frame, plural engaging grooves and plural air holes formed in the fitting frame, plural long positioning holes bored in two sides, and an inclined flexible contact strap located inside the top transversely. The eyeglass is combined with the outside of the flexible frame, provided with a curved recessed surface having the same shape as the fitting frame of the flexible frame, plural engaging projections corresponding to the engaging grooves of the flexible frame, and a connecting arms located at two sides respectively. Each of the connecting arms has two positioning bars used to interlock with the positioning holes of the flexible frame.

### BRIEF DESCRIPTION OF DRAWINGS

This invention is better understood by referring to the accompanying drawings, wherein:

FIG. 1 is an exploded perspective view of a preferred embodiment of a pair of collapsible goggles in the present invention;

FIG. 2 is an exploded perspective view of an eyeglass and a flexible frame in the preferred embodiment of a. pair of collapsible goggles in the present invention;

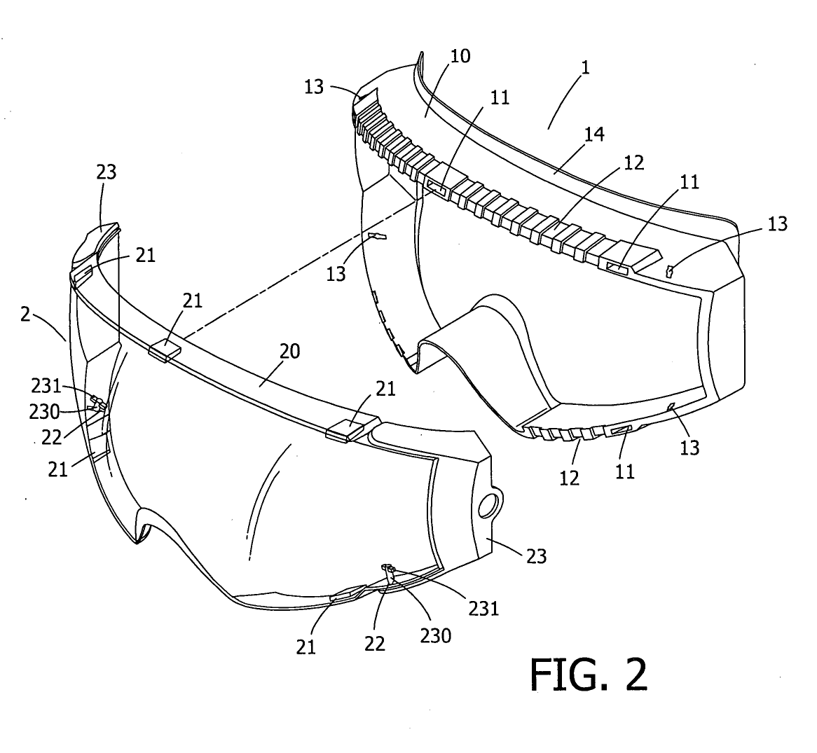

FIG. 3 is a perspective view of the preferred embodiment of a pair of collapsible goggles in the present invention; and

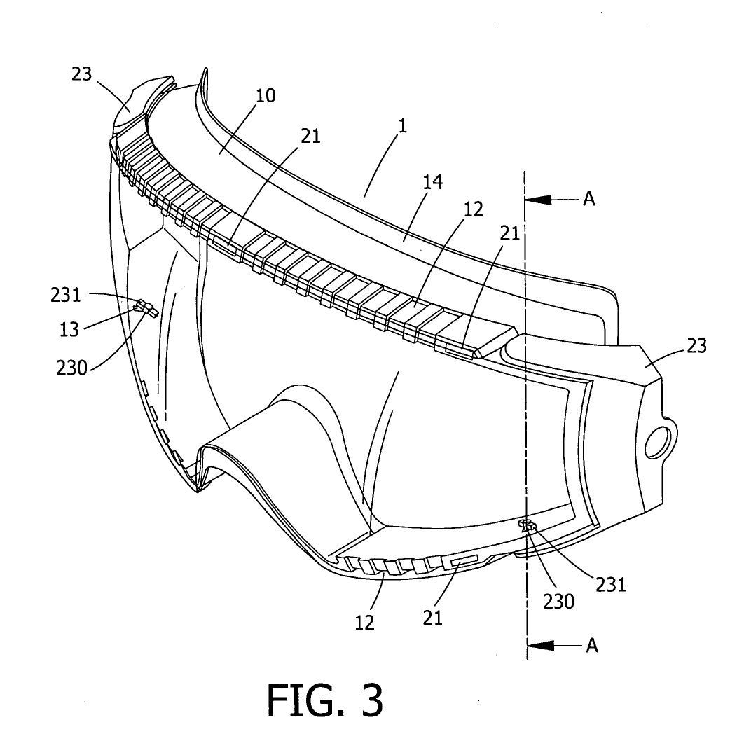

FIG. 4 is a cross-sectional view of A-A line in FIG. 3.

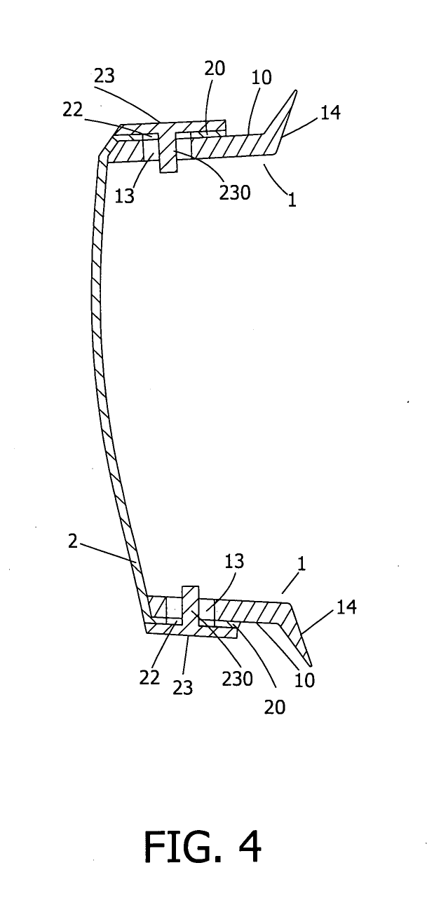

### DETAILED DESCRIPTION OF THE PREFERRED EMBODIMENT

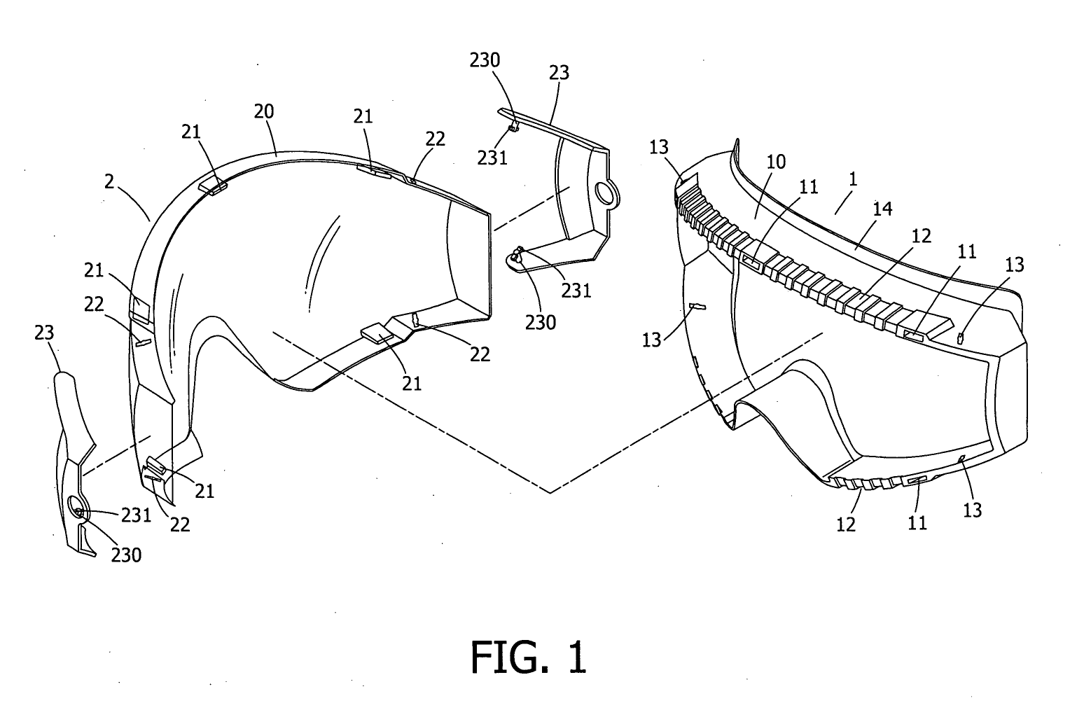

As shown in FIGS. 1˜4, a preferred embodiment of a pair of collapsible goggles in the present invention consists of a flexible frame 1 and an eyeglass 2.

The flexible frame 1 is formed mostly waved, provided with a fitting frame 10, plural engaging grooves 11 and plural air holes 12 formed around the front edge, plural long positioning holes 13 bored in two sides, and an inclined flexible contact strap 14 located inside the top transversely. So a wearer of the goggles may feel comfortable in using, as the inclined flexible contact trap softly contacts with the skin of the wearer's face.

The eyeglass 2 is combined outward with the flexible frame 1, shaped the same as the fitting frame 10 of the flexible frame 1. The eyeglass 2 is provided with a curved recessed surface 20 employed to wrap up outward the fitting frame 10, and two connecting arms 23 installed at two sides. Formed around the curved recessed surface 20 are plural engaging projections 21 corresponding to the engaging grooves 11 of the flexible frame 1 and plural joint holes 22. The engaging projections 21 have a size slightly larger than that of the engaging grooves 11. Each of the connecting arms 23 has a positioning bar 230 with a locking end 231 formed at two sides respectively, with the locking end 231 formed not facing to the positioning hole 13 of the flexible frame 1.

In assembly, the fitting frame 10 of the flexible frame 1 is first fitted in the curved recessed surface 20 of the eyeglass 2, with the engaging projections 21 of the eyeglass 2 inserted in the engaging grooves 11. Next, the positioning bars 230 of the connecting arms 23 are inserted through the joint holes 22 to further engage with the positioning hole 13. By the time, fixed by the engaging projections 21 of the eyeglass 2, the fitting frame 10 of the flexible frame 1 can thus be rigidly combined with the eyeglass 2, with the locking ends 231 of the positioning bars 230 obliquely interlocked with the positioning holes 13 to prevent them from separating from each other.

The invention has the following advantages as can be seen from the foresaid description.

1. The flexible frame 1 can be fixedly combined with the eyeglass 2 completely.

2. The eyeglass 2 has the surface maintained smooth and the flexible contact strap 14 enable a user to feel comfortable and secure while wearing the goggles of the invention.

3. It is pretty easy and convenient to combine the flexible frame 1 with the eyeglass 2.

4. As the flexible frame 1 and the eyeglass 2 are totally fixedly combined together by the engaging projections 21, they are assembled as an integral whole.

While the preferred embodiment of the invention has been described above, it will be recognized and understood that various modifications may be made therein and the appended claims are intended to cover all such modifications that may fall within the spirit and scope of the invention.

What is claimed is:

1. A pair of collapsible goggles comprising:
a flexible frame formed mostly waved and provided with a fitting frame, plural engaging grooves and plural air holes formed around a front edge of said fitting frame, plural long positioning holes bored in two sides of said flexible frame, an inclined flexible contact strap located inside a top of said flexible frame; and
an eyeglass combined with an outside of said flexible frame and provided with a curved recessed surface that is shaped the same as said fitting frame of said flexible frame, plural engaging projections formed around said curved recessed surface corresponding to said engaging grooves of said flexible frame, two connecting arms installed at two sides of said flexible frame and respectively having two positioning bars employed to interlock fixedly with said positioning holes of said flexible frame.

2. The pair of collapsible goggles as claimed in claim 1, wherein each of said positioning bars of said connecting arms has a free end formed with a locking end located not facing to said positioning hole of said flexible frame so as to prevent them from separating from each other.

3. The pair of collapsible goggles as claimed in claim 1, wherein said fitting frame of said flexible frame is completely attached to said curved recessed surface of said eyeglass so that said engaging grooves of said fitting frame can be engaged with said engaging projections of said eyeglass to make said flexible frame extended straight.
